(19) United States
(12) Patent Application Publication
(10) Pub. No.: US 2018/0180993 A1
(43) Pub. Date: Jun. 28, 2018

(54) NEW TRIAZINE AS PHOTO INITIATORS AND THEIR PREPARATION

(71) Applicant: Covestro Deutschland AG, Leverkusen (DE)

(72) Inventors: Koichi KAWAMURA, Odenthal (DE); Thomas RÖLLE, Leverkusen (DE); Dennis HÖNEL, Zülpich-Wichterich (DE); Friedrich-Karl BRUDER, Krefeld (DE)

(21) Appl. No.: 15/738,867

(22) PCT Filed: Jun. 21, 2016
(86) PCT No.: PCT/EP2016/064313

§ 371 Date: Dec. 21, 2017

(30) Foreign Application Priority Data
Jun. 23, 2015 (EP) 15173236.9

(51) Int. Cl. G03F 7/031; C07D 251/24; G03F 7/00; G03F 7/035; G03H 1/02

(52) U.S. Cl. CPC G03F 7/031; C07D 251/24; G11B 7/24044; G03F 7/035; G03H 1/02; G03F 7/001

(57) ABSTRACT

The present invention relates to new triazine photoinitiators, a new process for their preparation, and a photopolymer composition comprising a photopolymerizable component and the new triazine photoinitiators. Further aspects of the present invention are a photopolymer comprising said photopolymer composition, a holographic medium which comprises such a photopolymer, a hologram comprising the holographic medium, and a device such a display, chip card, security document, bank note and/or holographic optical element comprising said hologram.

The present invention relates to new triazine photoinitiators, a new process for their preparation, and a photopolymer composition comprising a photopolymerizable component and the new triazine photoinitiators. Further aspects of the present invention are a photopolymer comprising said photopolymer composition, a holographic medium which comprises such a photopolymer, a hologram comprising the holographic medium, and a device such a display, chip card, security document, bank note and/or holographic optical element comprising said hologram.

Photopolymers which can especially be used to make holographic media are known in the art. WO 2012/062655 A2 for example discloses photopolymers which comprise three dimensional cross linked polyurethane matrix polymers, acrylate writing monomers and a photo initiator system. Holographic media made from these photopolymers show excellent holographic performance.

The holographic performance of photopolymer is decisively determined by the refractive index modulation Δn produced in the photopolymer by holographic exposure. In holographic exposure, the interference field of signal light beam and reference light beam (in the simplest case, that of two plane waves) is mapped into a refractive index grating by the local photopolymerization of, for example, high refractive index acrylates at loci of high intensity in the interference field. The refractive index grating in the photopolymer (the hologram) contains all the information of the signal light beam. Illuminating the hologram with only the reference light beam will then reconstruct the signal. The strength of the signal thus reconstructed relative to the strength of the incident reference light is diffraction efficiency, DE in what follows.

In the simplest case of a hologram resulting from the superposition of two plane waves, the DE is the ratio of the intensity of the light diffracted on reconstruction to the sum total of the intensities of the incident reference light and the diffracted light. The higher the DE, the greater the efficiency of a hologram with regard to the amount of reference light needed to visualize the signal with a fixed brightness.

When the hologram is illuminated with white light, for example, the width of the spectral range which can contribute to reconstructing the hologram is likewise only dependent on the layer thickness d. The relationship which holds is that the smaller the thickness d, the greater the particular spectral bandwidth will be. Therefore, to produce bright and easily visible holograms, it is generally desirable to seek a high Δn and a low thickness d while maximizing DE. That is, increasing Δn increases the latitude to engineer the layer thickness d without loss of DE for bright holograms. Therefore, the optimization of Δn is of outstanding importance in the optimization of photopolymer formulations (P. Hariharan, Optical Holography, 2^{nd} Edition, Cambridge University Press, 1996).

Another important property of photopolymers for holographic media is their sensitivity to light which is used during the writing process. As the light intensity of light sources suitable for hologram recording is limited by the availability of such lasers it is desirable to provide photopolymers with a high sensitivity, i.e. photopolymers into which holograms can be recorded with the lowest possible light intensity.

U.S. Pat. No. 5,489,499 B1 describes 2-cinnamyl-4-trichloromethyl-6-trifluoromethyl-s-triazines in photopolymerizable compositions, where they are used in polymer compositions comprising aromatic meth acrylates and meth acrylic acid with methyl celluloses acetate in the reduction of coloration of products of decomposition by exposure to light and coloration due to exposure to light during storage in bright places.

It was thus an object of the present invention to provide a photoinitiators for the preparation of photopolymers for holographic media having a higher sensitivity compared to known holographic media.

EP 1457190 A1 describes bis(trichloromethyl)triazines as initiators for photopolymerization. The preparation of the respective bis(trichloromethyl)triazines, however, involves the use of HCl gas and preferably an additional strong Lewis acid, as e.g. disclosed in Wakabayashi, Ko; Tsunoda, Masaru; Suzuki, Yasushi, Bulletin of the Chemical Society of Japan (1969), 42 (10), 2924-31, which is tedious in work-up and unfavorable in handling.

It was therefore a further objective of the present invention to provide a new, versatile and economic process for preparing said photoinitiators while avoiding the use of HCl gas.

This first objective was achieved by providing a triazine photoinitiator of the formula (I)

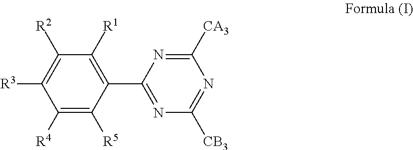

in which

A independently represents halogen,

B independently represents halogen different from A,

R¹-R⁵ independently represent hydrogen, halogen, alkyl, alkoxy, alkenyl, alkynyl, alkylthio, alkylseleno, nitro group with R¹ and R² and/or R² and R³ and/or R³ and R⁴ and/or R⁴ and R⁵ optionally form a 3 to 5 membered saturated or unsaturated ring which is optionally substituted with up to 2 hetero atoms and/or COOR⁶, COR⁷, CONHR⁸ radicals.

R⁶, R⁷, R⁸ all independently from one another represent hydrogen, halogen and/or C₁-C₁₀-alkyl and/or C₁-C₁₀-alkoxy-substituted linear C₅-C₂₀-alkyl, in which up to 6 carbon atoms may be substituted with oxygen with the prerequisite that each two oxygen atoms are entangled by at least 2 carbon atoms and R⁶, R⁷, R⁸ start with at least 2 carbon atoms and the terminal group of the C₅-C₂₀-alkyl group is a methyl group.

In an embodiment of the invention, A represents a Cl atom and B represents a F atom.

A further object of the invention is a process for the preparation of a triazine according to formula (I) comprising the steps of
a. Reacting the respective benzamidine hydrochloride of formula (II) with trihalogeno acetonitrile in the presence of a catalyst and

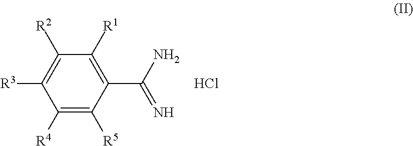

b. Reacting the resulting N-(benzamidyl) trihalogeno-acetamidine with trihalogeno acetic anhydride, whereby the radicals of R¹ to R⁵ are those as defined for formula (I) above.

In an embodiment of the inventive process, the trihalogeno-acetonitrile of a) carries three halogen atoms different from the three halogen atoms of the trihalogeno-acetic anhydride of b).

It has been found for the first time that the novel bis (trihalomethyl)-s-triazines characterized by the formula (I) show as good a photosensitivity as conventional bis (trichloromethyl)-s-triazines in photo-polymerization.

It has further been found that photopolymers with a photo initiator comprising a compound according to formula (I) can be used to make photopolymer compositions and holographic media with a very high sensitivity to light. Furthermore, it was found that they can be synthesized easily avoiding the use of HCl gas.

In one embodiment of the inventive new triazines according to formula (I), R³ represents a hydrogen, methyl, halogene, methoxy, cyano, carboxylate, alkoxycarbonyl, nitro or a trihalogenomethyl radical, while R¹, R², R⁴ and R⁵ independently represent hydrogen, halogen, alkyl, alkoxy, alkenyl, alkynyl, alkylthio, alkylseleno, nitro group with R¹ and R² and/or R² and R³ and/or R³ and R⁴ and/or R⁴ and R⁵ optionally form a 3 to 5 membered saturated or unsaturated ring which is optionally substituted with up to 2 hetero atoms and/or COOR⁶, COR⁷, CONHR⁸ radicals while R⁶, R⁷, R⁸ represent all independently from one another hydrogen, halogen and/or C₁-C₁₀-alkyl and/or C₁-C₁₀-alkoxy-substituted linear C₅-C₂₀-alkyl, in which up to 6 carbon atoms may be substituted with oxygen with the prerequisite that each two oxygen atoms are entangled by at least 2 carbon atoms and R⁶, R⁷, R⁸ start with at least 2 carbon atoms and the terminal group of the C₅-C₂₀-alkyl group is a methyl group.

In an embodiment of the invention R¹, R², R⁴ and R⁵ represent hydrogen. In another embodiment, R¹, R², R⁴ and R⁵ represent hydrogen and R³ represents a hydrogen, methyl, fluorine or methoxy radical.

In a further embodiment of the invention, A represents a Cl atom and B represents a F atom.

In a preferred embodiment of the inventive process as described above, the benzamidine hydrochloride is reacted with trichloroacetonitrile and the resulting N-(benzamidyl) trichloro acetamidine is reacted with trifluoroacetic anhydride.

In another embodiment of the invention, it is preferred to use reaction temperatures at step a from 0 to 50° C., especially preferred from 0 to 30° C.

In a further embodiment, the use of reaction temperatures at step b from −10 to 150° C. is preferred, specially preferred is a reaction temperature from 0 to 80° C.

It is preferred to use alcoholic solvents during step a). It is especially preferred to use methanol and/or ethanol and/or the isomeric propanols during step a).

It is preferred to use aprotic solvents during step b). It is further preferred to use diethyl ether, tetrahydrofuran, dimethoxyethane, benzene, toluene, chloroform, tetrachloromethan, dichloromethane. It is especially preferred to use tetrahydrofuran or chloroform.

Preferred benzamidine hydrochloride as starting material for the inventive process as described above are those of formula (II)

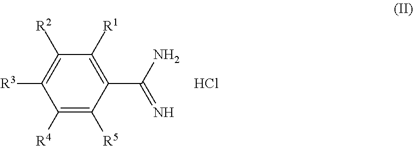

whereby the radicals R¹ to R⁵ have the meaning of those as defined in Formula (I) above. Preferably, R¹ to R⁵ represent independently methyl, ethyl, linear or branched or cyclic or substituted C₃-C₁₀ alkyl, chlorine, fluorine, aryl, hetero aryl, nitrile or COOR⁶, COR⁷ CONHR⁸ radicals, whereby R⁶, R⁷, R⁸ independently represent hydrogen, halogen and/or C₁-C₁₀-alkyl and/or C₁-C₁₀-alkoxy-substituted linear C₅-C₂₀-alkyl, in which up to 6 carbon atoms may be substituted with oxygen with the prerequisite that each two oxygen atoms are entangled by at least 2 carbon atoms and R⁶, R⁷, R⁸ start with at least 2 carbon atoms and the terminal group of the C₅-C₂₀-alkyl group is a methyl group.

Preferred starting compounds are unsubstitutes benzamidine hydrochloride, 4-fluoro benzamidine hydrochloride, 4-methyl benzamidine hydrochloride, 4-methoxy benzamidine hydrochloride, 4-cyano benzamidine hydrochloride, 4-alkoxycarbonyl benzamidine hydrochloride, 4-carboxylate benzamidine hydrochloride, 4-halogeno benzamidine hydrochloride, 4-nitro benzamidine hydrochloride and 4-trihalogenomethyl benzamidine hydrochloride, especially preferred are unsustitutes benzamidine hydrochloride, 4-fluoro benzamidine hydrochloride, 4-methyl benzamidine hydrochloride, and 4-methoxy benzamidine hydrochloride.

Catalysts as used in step a of the process are generally describes as bases to catch the hydrochloride component such as alkali bases, especially alkali hydroxides, preferably LiOH, NaOH, KOH, earth alkaline bases such as earth alkaline hydroxides, especially Ca(OH)₂, alkali metal alcoxides, preferably NaOMe, or amine bases such as tertiary amines.

A further object of the invention is a photopolymer composition comprising the inventive photoinitiators of formula (I).

The photopolymer composition may comprise a photopolymerizable component and a photo initiator system, whereby the photo initiator system comprises a triazine photoinitiator as described above or a triazine obtainable according to the process described above.

In an embodiment the photopolymer composition may comprise a photopolymerizable component and a photo initiator system, whereby the photo initiator system comprises a triazine photoinitiator as described above.

In another embodiment the photopolymer composition may comprise a photopolymerizable component and a photo initiator system, whereby the photo initiator system comprises a triazine obtainable according to the process described above.

In a preferred embodiment, the photopolymer composition may comprise 0.01 to 20.00 weight-%, preferably 0.2 to 15 weight-% and most preferably 0.5 to 10 weight-% of the compound according to formula (I). If this value falls below 0.01% by weight, the resulting photosensitive material exhibits insufficient sensitivity.

The photopolymer composition according to the present invention may further comprise a spectral sensitizing dye for adjusting the wavelength to which it is sensitive in addition to the ethylenically unsaturated compound and the inventive trihalomethyl-s-triazine of formula (I). As such, various spectral sensitizing dye compounds known in the art may be used. For the details of these specific sensitizing dyes, reference can be made to the foregoing patents concerning photopolymerization initiator, Research Disclosure, Vol. 200, December 1980, Item 20036, and Katsumi Tokumaru & Shin Oogawara, “Sensitizer”, Kodansha, 1987, pp. 160-163 or WO 2012/062655 A2.

The amount of the spectral sensitizing dye is generally from 0.001 to 10% by weight, preferably from 0.02 to 2% by weight, particularly preferably from 0.1 to 1% by weight, based on the total solid content of the photopolymer composition of the present invention.

The photopolymer composition according to the present invention may further comprise auxiliaries for accelerating the polymerization thereof, a reducing agent such as an oxygen remover, and a chain transfer agent for an active hydrogen donor or other compounds for accelerating the polymerization thereof according to a chain transfer reaction. Examples of the oxygen remover include phosphine, phosphonate, phosphite, stannous salt, and other compounds which can be easily oxidized by oxygen. Specific examples of such compounds include N-phenylglycine, trimethylbarbiturtic acid, N,N-dimethyl-2,6-diisopropylaniline, and N,N,N-2,4,6-pentamethylaniline. Further, thiols, thioketones, trihalomethyl compounds, lophine dimer compounds, iodonium salts, sulfonium salts, azinium salts and organic peroxides as mentioned below are useful as polymerization accelerators.

The photopolymerizable component may further comprise a thermal polymerization inhibitor as necessary. The thermal polymerization inhibitor is adapted to inhibit the thermal polymerization of a photopolymerizable composition or the polymerization thereof with time. With such a thermal polymerization inhibitor, the chemical stability of the photo-polymerizable composition during the preparation or storage thereof can be enhanced.

The photo initiator system may preferably further comprise at least one co-initiator, selected from carbonyl initiators, borate initiators, trichloromethyl initiators, aryloxide initiators, bisimidazole initiators, ferrocene initiators, aminoalkyl initiators, oxime initiator, thiol initiators, peroxide intiators. Examples of the co-initiator include carbonyl compounds such as benzoin ethyl ether, benzophenone, and diethoxyacetophenone; acylphosphine oxide compounds such as 2,4,6-trimethylbenzoyldiphenylphosphine oxide and bis(2,4,6-trimethylbenzoyl)phenylphosphine oxide; organic tin compounds such as tributylbenzyltin; alkylaryl borates such as tetrabutylammonium triphenylbutylborate, tetrabutylammonium tris(tert-butylphenyl)butylborate, and tetrabutylammonium trinaphtylbutylborate; diaryliodonium salts such as diphenyliodonium hexafluorophosphate, diphenyliodonium tetrafluoroborate, and diphenyliodonium hexafluoroantimonate; iron arene complexes such as (η5-cyclopentadienyl)(η6-cumenyl)-iron hexafluorophosphate; triazine compounds such as tris(trichloromethyl)triazine; organic peroxides such as 3,3′-di(tert-butylperoxycarbonyl)-4,4′-di(methoxycarbonyl)benzophenone, 3,3′,4,4′-tetrakis(tert-butylperoxycarbonyl)benzophenone, di-tert-butylperoxyisophthalate, 2,5-dimethyl-2,5-bis(benzoylperoxy)hexane, and tert-butylperoxy benzoate; and bis-imidazole derivatives such as 2,2′-bis(o-chlorophenyl)-4,4′,5,5′-tetraphenyl-1,1′-bis-imidazole.

According to still another preferred embodiment the photopolymer composition may further comprises matrix polymers. The matrix polymers may especially be three dimensional cross-linked and more preferably three dimensional cross-linked polyurethanes.

Such three dimensional cross-linked polyurethanes matrix polymers can for example be obtained by reacting a polyisocyanate component a) and an isocyanate-reactive component b).

The polyisocyanate component a) comprises at least one organic compound having at least two NCO groups. These organic compounds may especially be monomeric di- and triisocyanates, polyisocyanates and/or NCO-functional prepolymers. The polyisocyanate component a) may also contain or consist of mixtures of monomeric di- and triisocyanates, polyisocyanates and/or NCO-functional prepolymers.

Monomeric di- and triisocyanates used may be any of the compounds that are well known per se to those skilled in the art, or mixtures thereof. These compounds may have aromatic, araliphatic, aliphatic or cycloaliphatic structures. The monomeric di- and triisocyanates may also comprise minor amounts of monoisocyanates, i.e. organic compounds having one NCO group.

Examples of suitable monomeric di- and triisocyanates are butane 1,4-diisocyanate, pentane 1,5-diisocyanate, hexane 1,6-diisocyanate (hexamethylene diisocyanate, HDI), 2,2,4-trimethylhexamethylene diisocyanate and/or 2,4,4-trimethylhexamethylene diisocyanate (TMDI), isophorone diisocyanate (IPDI), 1,8-diisocyanato-4-(isocyanatomethyl)octane, bis(4,4′-isocyanatocyclohexyl)methane and/or bis(2′,4-isocyanatocyclohexyl)methane and/or mixtures thereof having any isomer content, cyclohexane 1,4-diisocyanate, the isomeric bis(isocyanatomethyl)cyclohexanes, 2,4- and/or 2,6-diisocyanato-1-methylcyclohexane (hexahydrotolylene 2,4- and/or 2,6-diisocyanate, H₆-TDI), phenylene 1,4-diisocyanate, tolylene 2,4- and/or 2,6-diisocyanate (TDI), naphthylene 1,5-diisocyanate (NDI), diphenylmethane 2,4′- and/or 4,4′-diisocyanate (MDI), 1,3-bis(isocyanatomethyl)benzene (XDI) and/or the analogous 1,4 isomers or any desired mixtures of the aforementioned compounds.

Suitable polyisocyanates are also compounds which have urethane, urea, carbodiimide, acylurea, amide, isocyanurate, allophanate, biuret, oxadiazinetrione, uretdione and/or iminooxadiazinedione structures and are obtainable from the aforementioned di- or triisocyanates.

More preferably, the polyisocyanates are oligomerized aliphatic and/or cycloaliphatic di- or triisocyanates, it being possible to use especially the above aliphatic and/or cycloaliphatic di- or triisocyanates.

Very particular preference is given to polyisocyanates having isocyanurate, uretdione and/or iminooxadiazinedione structures, and biurets based on HDI or mixtures thereof.

Suitable prepolymers contain urethane and/or urea groups, and optionally further structures formed through modification of NCO groups as specified above. Prepolymers of this kind are obtainable, for example, by reaction of the abovementioned monomeric di- and triisocyanates and/or polyisocyanates a1) with isocyanate-reactive compounds b1).

Isocyanate-reactive compounds b1) used may be alcohols, amino or mercapto compounds, preferably alcohols. These may especially be polyols. Most preferably, isocyanate-reactive compounds b1) used may be polyester polyols, polyether polyols, polycarbonate polyols, poly(meth)acrylate polyols and/or polyurethane polyols.

Suitable polyester polyols are, for example, linear polyester diols or branched polyester polyols, which can be obtained in a known manner by reaction of aliphatic, cycloaliphatic or aromatic di- or polycarboxylic acids or anhydrides thereof with polyhydric alcohols of OH functionality ≥2. Examples of suitable di- or polycarboxylic acids are polybasic carboxylic acids such as succinic acid, adipic acid, suberic acid, sebacic acid, decanedicarboxylic acid, phthalic acid, terephthalic acid, isophthalic acid, tetrahydrophthalic acid or trimellitic acid, and acid anhydrides such as phthalic anhydride, trimellitic anhydride or succinic anhydride, or any desired mixtures thereof. The polyester polyols may also be based on natural raw materials such as castor oil. It is likewise possible that the polyester polyols are based on homo- or copolymers of lactones, which can preferably be obtained by addition of lactones or lactone mixtures, such as butyrolactone, ε-caprolactone and/or methyl-ε-caprolactone onto hydroxy-functional compounds such as polyhydric alcohols of OH functionality ≥2, for example of the abovementioned type.

Examples of suitable alcohols are all polyhydric alcohols, for example the C₂-C₁₂ diols, the isomeric cyclohexanediols, glycerol or any desired mixtures thereof.

Suitable polycarbonate polyols are obtainable in a manner known per se by reaction of organic carbonates or phosgene with diols or diol mixtures.

Suitable organic carbonates are dimethyl, diethyl and diphenyl carbonate.

Suitable diols or mixtures comprise the polyhydric alcohols of OH functionality ≥2 mentioned per se in the context of the polyester segments, preferably butane-1,4-diol, hexane-1,6-diol and/or 3-methylpentanediol. It is also possible to convert polyester polyols to polycarbonate polyols.

Suitable polyether polyols are polyaddition products, optionally of blockwise structure, of cyclic ethers onto OH- or NH-functional starter molecules.

Suitable cyclic ethers are, for example, styrene oxides, ethylene oxide, propylene oxide, tetrahydrofuran, butylene oxide, epichlorohydrin, and any desired mixtures thereof.

Starters used may be the polyhydric alcohols of OH functionality ≥2 mentioned per se in the context of the polyester polyols, and also primary or secondary amines and amino alcohols.

Preferred polyether polyols are those of the aforementioned type based exclusively on propylene oxide, or random or block copolymers based on propylene oxide with further 1 alkylene oxides. Particular preference is given to propylene oxide homopolymers and random or block copolymers containing oxyethylene, oxypropylene and/or oxybutylene units, where the proportion of the oxypropylene units based on the total amount of all the oxyethylene, oxypropylene and oxybutylene units amounts to at least 20% by weight, preferably at least 45% by weight. Oxypropylene and oxybutylene here encompasses all the respective linear and branched C₃ and C₄ isomers.

Additionally suitable as constituents of the polyol component b1), as polyfunctional, isocyanate-reactive compounds, are also low molecular weight (i.e. with molecular weights ≤500 g/mol), short-chain (i.e. containing 2 to 20 carbon atoms), aliphatic, araliphatic or cycloaliphatic di-, tri- or polyfunctional alcohols.

These may, for example, in addition to the abovementioned compounds, be neopentyl glycol, 2-ethyl-2-butylpropanediol, trimethylpentanediol, positionally isomeric diethyloctanediols, cyclohexanediol, 1,4-cyclohexanedimethanol, 1,6-hexanediol, 1,2- and 1,4-cyclohexanediol, hydrogenated bisphenol A, 2,2-bis(4-hydroxycyclohexyl)propane or 2,2-dimethyl-3-hydroxypropyl 2,2-dimethyl-3-hydroxypropionate. Examples of suitable triols are trimethylolethane, trimethylolpropane or glycerol. Suitable higher-functionality alcohols are di(trimethylolpropane), pentaerythritol, dipentaerythritol or sorbitol.

It is especially preferable when the polyol component is a difunctional polyether, polyester, or a polyether-polyester block copolyester or a polyether-polyester block copolymer having primary OH functions.

It is likewise possible to use amines as isocyanate-reactive compounds b1). Examples of suitable amines are ethylenediamine, propylenediamine, diaminocyclohexane, 4,4′-dicyclohexylmethanediamine, isophoronediamine (IPDA), difunctional polyamines, for example the Jeffamines®, amine-terminated polymers, especially having number-average molar masses ≤10 000 g/mol. Mixtures of the aforementioned amines can likewise be used.

It is likewise possible to use amino alcohols as isocyanate-reactive compounds b1). Examples of suitable amino alcohols are the isomeric aminoethanols, the isomeric aminopropanols, the isomeric aminobutanols and the isomeric aminohexanols, or any desired mixtures thereof.

All the aforementioned isocyanate-reactive compounds b1) can be mixed with one another as desired.

It is also preferable when the isocyanate-reactive compounds b1) have a number-average molar mass of ≥200 and ≤10 000 g/mol, further preferably ≥500 and ≤8000 g/mol and most preferably ≥800 and ≤5000 g/mol. The OH functionality of the polyols is preferably 1.5 to 6.0, more preferably 1.8 to 4.0.

The prepolymers of the polyisocyanate component a) may especially have a residual content of free monomeric di- and triisocyanates of <1% by weight, more preferably <0.5% by weight and most preferably <0.3% by weight.

It is optionally also possible that the polyisocyanate component a) contains, entirely or in part, organic compound whose NCO groups have been fully or partly reacted with blocking agents known from coating technology. Example of blocking agents are alcohols, lactams, oximes, malonic esters, pyrazoles, and amines, for example butanone oxime, diisopropylamine, diethyl malonate, ethyl acetoacetate, 3,5-dimethylpyrazole, ε-caprolactam, or mixtures thereof.

It is especially preferable when the polyisocyanate component a) comprises compounds having aliphatically bonded NCO groups, aliphatically bonded NCO groups being understood to mean those groups that are bonded to a primary carbon atom.

The isocyanate-reactive component b) preferably comprises at least one organic compound having an average of at least 1.5 and preferably 2 to 3 isocyanate-reactive groups. In the context of the present invention, isocyanate-reactive groups are regarded as being preferably hydroxyl, amino or mercapto groups.

The isocyanate-reactive component may especially comprise compounds having a numerical average of at least 1.5 and preferably 2 to 3 isocyanate-reactive groups.

Suitable polyfunctional, isocyanate-reactive compounds of the component b) are, for example, the above-described compounds b1), including all the preferred embodiments mentioned for the component b1).

Further examples of suitable polyethers and processes for preparation thereof are described in EP 2 172 503 A1, the disclosure of which in this regard is hereby incorporated by reference.

Reaction of the polyisocyanate component a) with the isocyanate-reactive component b) gives rise to a polymeric matrix material. More preferably, this matrix material is consisting of addition products of butyrolactone, ε-caprolactone and/or methyl-ε-caprolactone onto polyether polyols of a functionality of ≥1.8 and ≤3.1 having number-average molar masses of ≥200 and ≤4000 g/mol in conjunction with isocyanurates, uretdiones, iminooxadiazinediones and/or other oligomers based on HDI. Very particular preference is given to addition products of ε-caprolactone onto poly(tetrahydrofurans) having a functionality of ≥1.9 and ≤2.2 and number-average molar masses of ≥500 and ≤2000 g/mol, especially of ≥600 and ≤1400 g/mol, having a total number-average molar mass of ≥800 and ≤4500 g/mol, especially of ≥1000 and ≤3000 g/mol, in conjunction with oligomers, isocyanurates and/or iminooxadiazinediones based on HDI.

It is also possible that the photopolymer composition further comprises monomeric fluorourethanes and preferably monomeric fluorourethanes according to formula (III)

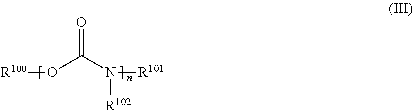

in which n is ≥1 and n is ≤8 and R¹⁰⁰, R¹⁰¹, R¹⁰² are hydrogen and/or, independently of one another, linear, branched, cyclic or heterocyclic organic rests which are unsubstituted or optionally also substituted by heteroatoms, at least one of the residues R¹⁰⁰, R¹⁰¹, R¹⁰² being substituted by at least one fluorine atom.

In a further preferred embodiment, the photopolymerizable component comprises or consists of at least one mono- and/or one multifunctional monomer. Further preferably, the photopolymerizable component may comprise or consist of at least one mono- and/or one multifunctional (meth)acrylate monomers. Most preferably, the photopolymerizable component may comprise or consist of at least one mono- and/or one multifunctional urethane (meth)acrylate.

Suitable acrylate monomers are especially compounds of the general formula (IV)

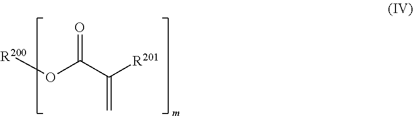

in which m≥1 and m≤4 and R²⁰⁰ is a linear, branched, cyclic or heterocyclic organic radical which is unsubstituted or else optionally substituted by heteroatoms and/or R²⁰¹ is hydrogen or a linear, branched, cyclic or heterocyclic organic radical which is unsubstituted or else optionally substituted by heteroatoms. More preferably, R²⁰⁰ is hydrogen or methyl and/or R²⁰¹ is a linear, branched, cyclic or heterocyclic organic radical which is unsubstituted or else optionally substituted by heteroatoms.

Acrylates and methacrylates refer, respectively, to esters of acrylic acid and methacrylic acid. Examples of acrylates and methacrylates usable with preference are phenyl acrylate, phenyl methacrylate, phenoxyethyl acrylate, phenoxyethyl methacrylate, phenoxyethoxyethyl acrylate, phenoxyethoxyethyl methacrylate, phenylthioethyl acrylate, phenylthioethyl methacrylate, 2-naphthyl acrylate, 2-naphthyl methacrylate, 1,4-bis(2-thionaphthyl)-2-butyl acrylate, 1,4-bis(2-thionaphthyl)-2-butyl methacrylate, bisphenol A diacrylate, bisphenol A dimethacrylate, and the ethoxylated analogue compounds thereof, N-carbazolyl acrylates.

Urethane acrylates mean compounds having at least one acrylic ester group and at least one urethane bond. Compounds of this kind can be obtained, for example, by reacting a hydroxy-functional acrylate or methacrylate with an isocyanate-functional compound.

Examples of isocyanate-functional compounds usable for this purpose are mono isocyanates, and the monomeric diisocyanates, triisocyanates and/or polyisocyanates mentioned under a). Examples of suitable monoisocyanates are phenyl isocyanate, the isomeric methylthiophenyl isocyanates. Di-, tri- or polyisocyanates have been mentioned above, and also triphenylmethane 4,4′,4″-triisocyanate and tris(p-isocyanatophenyl) thiophosphate or derivatives thereof with urethane, urea, carbodiimide, acylurea, isocyanurate, allophanate, biuret, oxadiazinetrione, uretdione, iminooxadiazinedione structure and mixtures thereof. Preference is given to aromatic di-, tri- or polyisocyanates.

Useful hydroxy-functional acrylates or methacrylates for the preparation of urethane acrylates include, for example, compounds such as 2-hydroxyethyl (meth)acrylate, polyethylene oxide mono(meth)acrylates, polypropylene oxide mono(meth)acrylates, polyalkylene oxide mono(meth)acrylates, poly(ε-caprolactone) mono(meth)acrylates, for example Tone® M100 (Dow, Schwalbach, Del.), 2-hydroxypropyl (meth)acrylate, 4-hydroxybutyl (meth)acrylate, 3-hydroxy-2,2-dimethylpropyl (meth)acrylate, hydroxypropyl (meth)acrylate, 2-hydroxy-3-phenoxypropyl acrylate, the hydroxy-functional mono-, di- or tetra-acrylates of polyhydric alcohols such as trimethylolpropane, glycerol, pentaerythritol, dipentaerythritol, ethoxylated, propoxylated or alkoxylated trimethylolpropane, glycerol, pentaerythritol, dipentaerythritol or the technical mixtures thereof. Preference is given to 2-hydroxyethyl acrylate, hydroxypropyl acrylate, 4-hydroxybutyl acrylate and poly(ε-caprolactone) mono(meth)acrylate.

It is likewise possible to use the fundamentally known hydroxyl-containing epoxy (meth)acrylates having OH contents of 20 to 300 mg KOH/g or hydroxyl-containing polyurethane (meth)acrylates having OH contents of 20 to 300 mg KOH/g or acrylated polyacrylates having OH contents of 20 to 300 mg KOH/g and mixtures thereof, and mixtures with hydroxyl-containing unsaturated polyesters and mixtures with polyester (meth)acrylates or mixtures of hydroxyl-containing unsaturated polyesters with polyester (meth)acrylates.

Preference is given especially to urethane acrylates obtainable from the reaction of tris(p-isocyanatophenyl) thiophosphate and/or m-methylthiophenyl isocyanate with alcohol-functional acrylates such as hydroxyethyl (meth)acrylate, hydroxypropyl (meth)acrylate and/or hydroxybutyl (meth)acrylate.

It is likewise possible that the photopolymerizable component comprises or consists of further unsaturated compounds such as α,β-unsaturated carboxylic acid derivatives, for example maleates, fumarates, maleimides, acrylamides, and also vinyl ethers, propenyl ethers, allyl ethers and compounds containing dicyclopentadienyl units, and also olefinically unsaturated compounds, for example styrene, α-methylstyrene, vinyltoluene and/or olefins.

However it is especially preferred if the photopolymerizable component comprises a mono- and/or multifunctional urethane-(meth)-acrylate.

The photopolymer composition may further comprise cationic polymerizable compounds such as cationic initiators, cationic polymerizable monomers or cationic polymerizable plasticizers as referred in US 20130034805A.

A further aspect of the invention is a photopolymer comprising an inventive photopolymer composition as described above. All embodiments as described above for the photopolymer composition shall apply for the photopolymer as well.

In an embodiment the photopolymer comprises a cross-linked network of matrix polymers, especially a three dimensional cross-linked network. In another embodiment, the photopolymer comprises polyurethane as matrix polymers.

In an embodiment, the photopolymer can be understood to be cured and/or end-reacted.

Another aspect of the present invention is a holographic media which comprises a photopolymer according to the invention.

The holographic media may contain or consist of the abovementioned photopolymer.

The holographic media may contain or consist of the abovementioned photopolymer composition.

The photopolymer can especially be used for production of holographic media in the form of a film. In this case, a ply of a material or material composite transparent to light within the visible spectral range (transmission greater than 85% within the wavelength range from 400 to 780 nm) as carrier is coated on one or both sides, and a cover layer is optionally applied to the photopolymer ply or plies.

The invention therefore also provides a process for producing a holographic medium, in which
(I) an inventive photopolymer is produced by mixing all the constituents,
(II) the photopolymer is converted to the form desired for the holographic medium at a processing temperature and
(III) cured in the desired form with urethane formation at a crosslinking temperature above the processing temperature.

Preferably, the photopolymer is produced in step I) by mixing the individual constituents.

Preferably, the photopolymer is converted in step II) to the form of a film. For this purpose, the photopolymer can be applied, for example, over the area of a carrier substrate, in which case, for example, the apparatuses known to those skilled in the art (doctor blade, knife-over-roll, comma bar, inter alia) or a slot die can be used. The processing temperature here can be in the range of 20 to 40° C., preferably in the range of 20 to 30° C.

The carrier substrate used may be a ply of a material or material composite transparent to light in the visible spectral range (transmission greater than 85% in the wavelength range from 400 to 800 nm).

Preferred materials or material composites for the carrier substrate are based on polycarbonate (PC), polyethylene terephthalate (PET), polybutylene terephthalate, polyethylene, polypropylene, cellulose acetate, cellulose hydrate, cellulose nitrate, cycloolefin polymers, polystyrene, polyepoxides, polysulphone, cellulose triacetate (CTA), polyamide, polymethylmethacrylate, polyvinyl chloride, polyvinyl butyral or polydicyclopentadiene or mixtures thereof. They are more preferably based on PC, PET and CTA. Material composites may be film laminates or coextrudates. Preferred material composites are duplex and triplex films formed according to one of the schemes A/B, A/B/A or A/B/C. Particular preference is given to PC/PET, PET/PC/PET and PC/TPU (TPU=thermoplastic polyurethane).

As an alternative to the aforementioned carrier substrates, it is also possible to use planar glass panes, which find use especially for large-area, high-accuracy exposures, for example for holographic lithography (Holographic interference lithography for integrated optics, IEEE Transactions on Electron Devices (1978), ED-25(10), 1193-1200, ISSN:0018-9383).

The materials or material composites of the carrier substrate may be given an antiadhesive, antistatic, hydrophobized or hydrophilized finish on one or both sides. The modifications mentioned serve the purpose, on the side facing the photopolymer, of making the photopolymer detachable without destruction from the carrier substrate. Modification of the opposite side of the carrier substrate from the photopolymer serves to ensure that the inventive media satisfy specific mechanical demands which exist, for example, in the case of processing in roll laminators, especially in roll-to-roll processes.

The carrier substrate may be coated on one or both sides.

The invention also provides a holographic medium obtainable by the process according to the invention.

The invention further provides a laminate structure comprising a carrier substrate, an inventive holographic medium applied thereto, and optionally a cover layer applied to the opposite side of the holographic medium from the carrier substrate.

The laminate structure may especially have one or more cover layers on the holographic medium in order to protect it from soil and environmental influences. For this purpose, it is possible to use polymer films or film composite systems, or further clearcoats.

The cover layers used are preferably film materials analogous to the materials used in the carrier substrate, and these may have a thickness of typically 5 to 200 μm, preferably 8 to 125 μm, more preferably 10 to 50 μm.

Preference is given to cover layers having a very smooth surface. A measure used here is the roughness, determined to DIN EN ISO 4288 “Geometrical Product Specifications (GPS)—Surface texture . . . ”, test condition: R3z front and reverse sides. Preferred roughnesses are in the region of less than or equal to 2 μm, preferably less than or equal to 0.5 μm.

The cover layers used are preferably PE or PET films of thickness 20 to 60 μm. More preferably, a polyethylene film having a thickness of 40 μm is used.

It is likewise possible that, in the case of a laminate structure on the carrier substrate, a further cover layer is applied as a protective layer.

In a preferred embodiment of the holographic media at least one hologram is recorded into it.

The inventive holographic media can be processed to holograms by means of appropriate recording processes for optical applications over the entire visible range (400-800 nm). Visual holograms include all holograms which can be recorded by methods known to those skilled in the art. These include in-line (Gabor) holograms, off-axis holograms, full-aperture transfer holograms, white light transmission holograms (“rainbow holograms”), Denisyuk holograms, off-axis reflection holograms, edge-lit holograms and holographic stereograms. Preference is given to reflection holograms, Denisyuk holograms, transmission holograms.

Another object of the invention is therefore a hologram comprising the inventive holographic medium as described above.

Possible optical functions of the holograms correspond to the optical functions of light elements such as lenses, mirrors, deflecting mirrors, filters, diffuser lenses, diffraction elements, light guides, waveguides, projection lenses and/or masks. These optical elements frequently have a frequency selectivity according to how the holograms have been exposed and the dimensions of the hologram.

In addition it is also possible to produce holographic images or representations, for example for personal portraits, biometric representations in security documents, or generally of images or image structures for advertising, security labels, brand protection, branding, labels, design elements, decorations, illustrations, collectable cards, images and the like, and also images which can represent digital data, including in combination with the products detailed above. Holographic images can have the impression of a three-dimensional image, but they may also represent image sequences, short films or a number of different objects according to the angle from which and the light source with which (including moving light sources) etc. they are illuminated. Because of this variety of possible designs, holograms, especially volume holograms, constitute an attractive technical solution for the abovementioned application.

Still another aspect of the present invention is a display comprising a holographic media according to the invention.

Examples for such displays are three dimensional displays, head-up displays, head-down displays in vehicles, displays in windows, on glasses, displays integrated in eye glasses.

Also the use of a holographic media according to the invention to make chip cards, security documents, bank notes and/or holographic optical elements especially for displays is an aspect of the present invention.

Therefore, another object of the invention is a device such as a display, chip card, security document, bank note and/or holographic optical element characterized in that it comprises a hologram according to the invention.

### EXAMPLES

The invention will be described in more detail by the following examples without restricting it thereto.

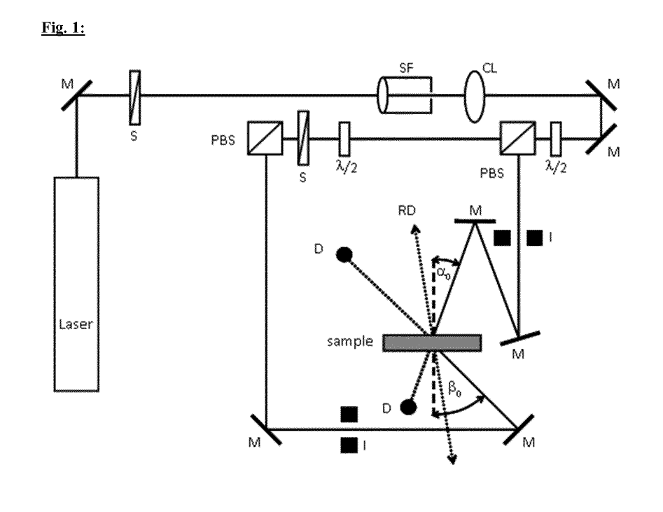

FIG. 1 shows the geometry of a holographic media tester (HMT) at λ=532 nm (DPSS laser).

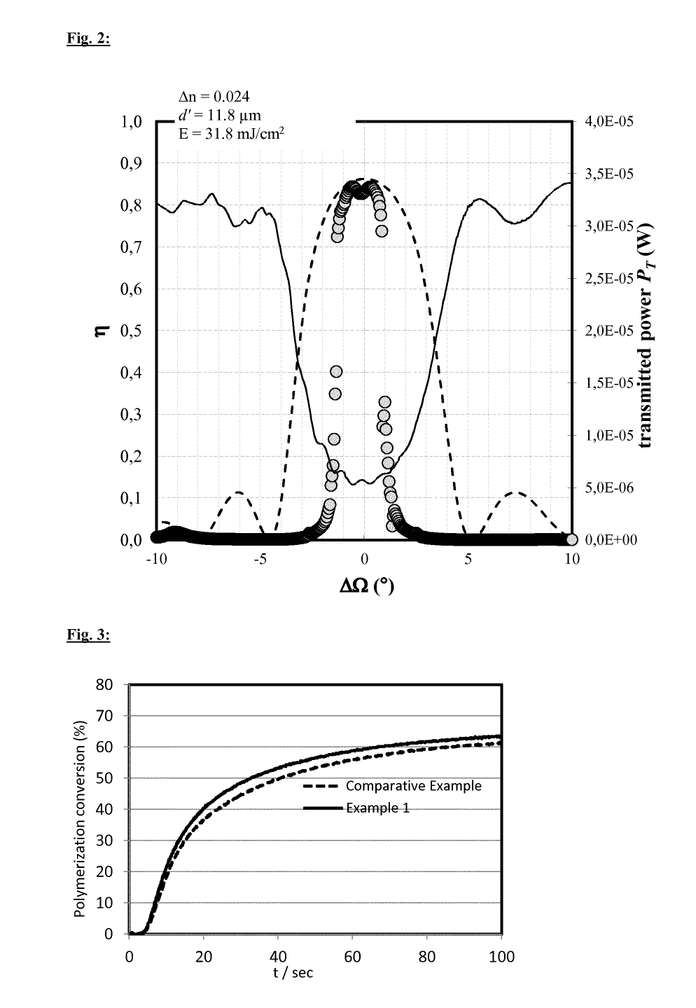

FIG. 2 shows the measured transmitted power P_{T} (right-hand y-axis) plotted as a solid line against the angle detuning ΔΩ and the measured diffraction efficiency η (left-hand y-axis) is plotted as filled circles against the angle detuning ΔΩ.

FIG. 3 shows the photopolymerization conversion in % of photopolymer formulations comprising Triazine 1 and Triazine TA against the irradiation exposure time t in sec.

### STARTING MATERIALS

Starting materials to synthesize compounds of formula (II) were prepared according to procedures reported in the literature or purchased.

The reagents and solvents used were acquired commercially.

| | |
|---|---|
| Trichloroacetonitrile | purchased from ABCR GmbH & CO. KG, |
| | Karlsruhe, Germany. |
| Trifluoroacetic | purchased from Sigma-Aldrich, Taufkirchen, |
| Anhydride | Germany. |
| Benzamidine*HCl | purchased from ABCR GmbH & CO. KG, |
| | Karlsruhe, Germany. |
| CGI-909 | Tetrabutylammonium tris(3-chloro-4-methylphenyl) |
| | (hexyl)borate, [1147315-11-4] is a product |
| | produced by BASF SE, Basle, Switzerland. |
| SR 349 | Ethoxylated (3) Bisphenol A Diacrylate, a product |
| | produced by Sartomer Americas, 502 Thomas Jones |
| | Way, Exton, PA 19341, USA. |
| Safranine O | purchased from Sigma-Aldrich, Taufkirchen, |
| | Germany. |
| Triazine-A | [3584-23-4] purchased from Midori Kagaku Co. |
| | Ltd, Tokyo Japan. Product no. TAZ-104. |
| Dye 1 | Preparation of Dye 1, 3H-Indolium, 2- |
| | [2-[4-[(2-chloroethyl)ethyl-amino]phenyl]ethenyl]- |
| | 1,3,3-trimethyl-, salt with 1-(2-ethylhexyl) 4- |
| | (1-ethylpentyl) 2-sulfobutanedioate (1:1) |
| | [1374689-58-3] is described in EP 2450893 A1. |
| Desmorapid Z | Dibutyltin dilaurate [77-58-7], product from |
| | Bayer MaterialScience AG, Leverkusen, Germany. |
| Desmodur ® | Product from Bayer MaterialScience AG, |
| N 3900 | Leverkusen, Germany, hexane diisocyanate-based |
| | polyisocyanate, iminooxadiazinedione content at |
| | least 30%, NCO content: 23.5%. |
| Fomrez UL 28 | Urethanization catalyst, commercial product of |
| | Momentive Performance Chemicals, Wilton, CT, |
| | USA. |

Test Methods:

Isocyanate Content (NCO Value)

The isocyanate contents reported were determined according to DIN EN ISO 11909.

Determination of Photo Sensitivity

The photosensitivity of the compounds was measured by preparing a photosensitive formulation as described below and measuring photopolymerization using FTIR. Thus, the photosensitive formulation was coated with a thickness of 25 μm onto a polyethylene film and covered with a further polyethylene film to prevent oxidation by oxygen from the air. The respective samples were measured by Real-Time FTIR (Vertex 70 FTIR spectrometer, Bruker Optik) using a 532 nm laser diode as an irradiation source with the irradiance intensity at the surface of the sample adjusted to 10 mW/cm². The kinetics of the polymerization was measured by following the decay of the acrylic double bond at 1635 cm⁻¹. The degree of acrylate double bond conversion C(%) was calculated from the decrease of the area of the IR absorption peak at 1635 cm⁻¹ of the sample after exposure using the following equation:

C(%)=(A₀−Aₜ)/A₀×100

A₀ represent the initial peak area before irradiation and Aₜ represent the peak area of the acrylic double bond at t time.

Holographic Testing:

Measurement of the Holographic Properties of Diffraction Efficiency DE and Refractive Index Contrast Δn of the Holographic Media by Means of Twin-Beam Interference in a Reflection Arrangement.

A holographic test setup as shown in FIG. 1 was used to measure the diffraction efficiency (DE) of the media. The beam of a DPSS laser (emission wavelength 532 nm) was converted to a parallel homogeneous beam with the aid of the spatial filter (SF) and together with the collimation lens (CL). The final cross sections of the signal and reference beam are fixed by the iris diaphragms (I). The diameter of the iris diaphragm opening is 0.4 cm. The polarization-dependent beam splitters (PBS) split the laser beam into two coherent beams of identical polarization. By means of the λ/2 plates, the power of the reference beam was set to 0.87 mW and the power of the signal beam to 1.13 mW. The powers were determined using the semiconductor detectors (D) with the sample removed. The angle of incidence (α₀) of the reference beam is −21.8°; the angle of incidence (β₀) of the signal beam is 41.8°. The angles are measured proceeding from the sample normal to the beam direction. According to FIG. 2, therefore, α₀ has a negative sign and β₀ a positive sign. At the location of the sample (medium), the interference field of the two overlapping beams produced a pattern of light and dark strips parallel to the angle bisectors of the two beams incident on the sample (reflection hologram). The strip spacing Λ, also called grating period, in the medium is ˜188 nm (the refractive index of the medium assumed to be ˜1.504).

FIG. 1 shows the geometry of a holographic media tester (HMT) at λ=532 nm (DPSS laser): M=mirror, S=shutter, SF=spatial filter, CL=collimator lens, λ/2=λ/2 plate, PBS=polarization-sensitive beam splitter, D=detector, I=iris diaphragm, α₀=−21.8°, β₀=41.8° are the angles of incidence of the coherent beams measured outside the sample (outside the medium). RD=reference direction of the turntable.

Holograms were recorded in the medium in the following manner:
Both shutters (S) are opened for the exposure time t.
Thereafter, with the shutters (S) closed, the medium is allowed 5 minutes for the diffusion of the as yet unpolymerized writing monomers.

The holograms recorded were then reconstructed in the following manner. The shutter of the signal beam remained closed. The shutter of the reference beam was opened. The iris diaphragm of the reference beam was closed to a diameter of <1 mm. This ensured that the beam was always completely within the previously recorded hologram for all angles of rotation (Ω) of the medium. The turntable, under computer control, swept over the angle range from Ωₘᵢₙ to Ωₘₐₓ with an angle step width of 0.05°. Ω is measured from the sample normal to the reference direction of the turntable. The reference direction of the turntable is obtained when the angles of incidence of the reference beam and of the signal beam have the same absolute value on recording of the hologram, i.e. α₀=−31.8° and β₀=31.8°. In that case, Ω_{recording}=0°. When α₀=−21.8° and β₀=41.8°, Ω_{recording} is therefore 10°. In general, for the interference field in the course of recording of the hologram:

α₀=θ₀+Ω_{recording}.

θ₀ is the semiangle in the laboratory system outside the medium and, in the course of recording of the hologram:

$${θ}_{0} = \frac{{α}_{0} - {β}_{0}}{2} .$$

Thus, in this case, θ₀=−31.8°. At each setting for the angle of rotation Ω, the powers of the beam transmitted in the zeroth order were measured by means of the corresponding detector D, and the powers of the beam diffracted in the first order by means of the detector D. The diffraction efficiency was calculated at each setting of angle Ω as the quotient of:

$$η = \frac{{P}_{D}}{{P}_{D} + {P}_{T}}$$

P_{D} is the power in the detector for the diffracted beam and P_{T} is the power in the detector for the transmitted beam.

By means of the process described above, the Bragg curve, which describes the diffraction efficiency η as a function of the angle of rotation Ω for the recorded hologram, was measured and saved on a computer. In addition, the intensity transmitted into the zeroth order was also recorded against the angle of rotation Ω and saved on a computer.

The maximum diffraction efficiency (DE=ηₘₐₓ) of the hologram, i.e. the peak value thereof, was determined at Ω_{reconstruction}. In some cases, it was necessary for this purpose to change the position of the detector for the diffracted beam in order to determine this maximum value.

The refractive index contrast Δn and the thickness d of the photopolymer layer were now determined by means of coupled wave theory (see: H. Kogelnik, The Bell System Technical Journal, Volume 48, November 1969, Number 9 page 2909-page 2947) from the measured Bragg curve and the variation of the transmitted intensity with angle. In this context, it should be noted that, because of the shrinkage in thickness which occurs as a result of the photopolymerization, the strip spacing Δ′ of the hologram and the orientation of the strips (slant) can differ from the strip spacing Δ of the interference pattern and the orientation thereof. Accordingly, the angle α₀′ and the corresponding angle of the turntable Ω_{reconstruction} at which maximum diffraction efficiency is achieved will also differ from α₀ and from the corresponding Ω_{recording}. This alters the Bragg condition. This alteration is taken into account in the evaluation process. The evaluation process is described hereinafter:

All geometric parameters which relate to the recorded hologram and not to the interference pattern are shown as parameters with primes.

For the Bragg curve η(Ω) of a reflection hologram, according to Kogelnik:

$$η = \{ \begin{matrix}\frac{1}{1 - \frac{1 - {( ξ / ν )}^{2}}{{sin}^{2} ( \sqrt{{ξ}^{2} - {ν}^{2}} )}} , & for {ν}^{2} - {ξ}^{2} < 0 \\ \frac{1}{1 + \frac{1 - {( ξ / ν )}^{2}}{{sin}^{2} ( \sqrt{{ν}^{2} - {ξ}^{2}} )}} , & for {ν}^{2} - {ξ}^{2} \geq 0\end{matrix} with \text{:} \\ ν + \frac{π \cdot Δ n \cdot {d}^{′}}{λ \cdot \sqrt{l \vert {c}_{s} \cdot {c}_{r} \vert}} ξ = - \frac{{d}^{′}}{2 \cdot {c}_{s}} \cdot DP \\ {c}_{s} = cos ( {ϑ}^{′} ) - cos ( {Ψ}^{′} ) \cdot \frac{λ}{n \cdot {Λ}^{′}} \\ {c}_{r} = cos ( {ϑ}^{′} ) \\ DP = \frac{π}{{Λ}^{′}} \cdot ( 2 \cdot cos ( {Ψ}^{′} - {ϑ}^{′} ) - \frac{λ}{n \cdot {Λ}^{′}} ) \\ {Ψ}^{′} = \frac{{β}^{′} + {α}^{′}}{2} {Λ}^{′} = \frac{λ}{2 \cdot n \cdot cos ( {Ψ}^{′} - {α}^{′} )}$$

In the reconstruction of the hologram, as explained analogously above:

ϑ′₀=θ₀+Ω

sin(ϑ′₀)=n·sin(ϑ′)

Under the Bragg condition, the “dephasing” DP=0. And it follows correspondingly that:

α′₀=θ₀+Ω_{reconstruction}

sin(α′₀)=n·sin(α′)

The as yet unknown angle β′ can be determined from the comparison of the Bragg condition of the interference field in the course of recording of the hologram and the Bragg condition in the course of reconstruction of the hologram, assuming that only shrinkage in thickness takes place. It then follows that:

$$sin ( {β}^{′} ) = \frac{1}{n} \cdot [ sin ( {α}_{0} ) + sin ( {β}_{0} ) - sin ( {θ}_{0} + {Ω}_{reconstruction} ) ]$$

v is the grating thickness, ξ is the detuning parameter and ψ′ is the orientation (slant) of the refractive index grating which has been recorded. α′ and β′ correspond to the angles α₀ and β₀ of the interference field in the course of recording of the hologram, except measured in the medium and applying to the grating of the hologram (after shrinkage in thickness). n is the mean refractive index of the photopolymer and was set to 1.504. λ is the wavelength of the laser light in the vacuum.

The maximum diffraction efficiency (DE=ηₘₐₓ), when ξ=0, is then calculated to be:

$$DE = {tanh}^{2} ( v ) = {tanh}^{2} ( \frac{π \cdot Δ n \cdot {d}^{′}}{λ \cdot \sqrt{cos ( {α}^{′} ) \cdot cos ( {α}^{′} - 2 Ψ )}} )$$

FIG. 2 shows the measured transmitted power P_{T} (right-hand y-axis) plotted as a solid line against the angle detuning ΔΩ; the measured diffraction efficiency η (left-hand y-axis) is plotted as filled circles against the angle detuning ΔΩ (to the extent allowed by the finite size of the detector), and the fitting to the Kogelnik theory as a broken line (left-hand y-axis).

The measured data for the diffraction efficiency, the theoretical Bragg curve and the transmitted intensity are, as shown in FIG. 2, plotted against the centered angle of rotation ΔΩ≡Ω_{reconstruction}−Ω=α′₀−ϑ′₀, also called angle detuning.

Since DE is known, the shape of the theoretical Bragg curve, according to Kogelnik, is determined only by the thickness d′ of the photopolymer layer. Δn is corrected via DE for a given thickness d′ such that measurement and theory for DE are always in agreement. d′ is adjusted until the angle positions of the first secondary minima of the theoretical Bragg curve correspond to the angle positions of the first secondary maxima of the transmitted intensity, and there is additionally agreement in the full width at half maximum (FWHM) for the theoretical Bragg curve and for the transmitted intensity.

Since the direction in which a reflection hologram also rotates when reconstructed by means of an Ω scan, but the detector for the diffracted light can cover only a finite angle range, the Bragg curve of broad holograms (small d′) is not fully covered in an Ω scan, but rather only the central region, given suitable detector positioning. Therefore, the shape of the transmitted intensity, which is complementary to the Bragg curve, is additionally employed for adjustment of the layer thickness d′.

FIG. 2 shows the plot of the Bragg curve η according to the coupled wave theory (broken line), the measured diffraction efficiency (filled circles) and the transmitted power (black solid line) against the angle detuning ΔΩ.

For a formulation, this procedure was repeated, possibly several times, for different exposure times t on different media, in order to find the mean energy dose of the incident laser beam in the course of recording of the hologram at which DE reaches the saturation value. The mean energy dose E is calculated as follows from the powers of the two component beams assigned to the angles α₀ and β₀ (reference beam where Pᵣ=0.87 mW and signal beam where Pₛ=1.13 mW), the exposure time t and the diameter of the iris diaphragm (0.4 cm):

$$E ( mJ / {cm}^{2} ) = \frac{2 \cdot [ {P}_{r} + {P}_{s} ] \cdot t ( s )}{π \cdot {0.4}^{2} {cm}^{2}}$$

The powers of the component beams were adjusted such that the same power density is attained in the medium at the angles α₀ and β₀ used.

In an alternative setup according to FIG. 1 a DPSS laser with an emission wavelength λ of 473 nm could be used. In this case α₀=−21.8° and β₀=41.8° are same as if using the emission wavelength λ=532 nm but the reference beam power was set to Pᵣ=1.31 mW and signal beam power was set to Pₛ=1.69 mW.

Preparation of Triazines:

### Synthesis of Triazine 1: 2-Phenyl-4-trichloromethyl-6-trifluoromethy-s-triazine

Step a

To a solution of 25.0 g of benzamidine hydrochloride and 100 mL of Methanol, 33.2 g of 25% solution of NaOMe were added drop wise with stirring at zero degree. After addition, the mixture was stirred for 30 min, then 22.2 g of trichloroacetonitrile were added at zero degree during 30 min. After the addition, the cooling bath was removed and the reaction mixture was continued to stir overnight. 100 mL of ethyl acetate was added to the reaction mixture and the solid that precipitates in the flask was removed by filtration. The filtered solution was evaporated and 300 mL of cyclohexane was added. After heating the mixture to reflux for 30 min, upper part of solution was removed by decantation and lower part was evaporated to yields N-(benzamidyl) trichloroacetamidine as oil. Yields 29.2 g.

Step b

10.0 g of N-(benzamidyl) trichloroacetamidine in 10 mL of tetrahydrofuran were added drop wise carefully to a cooed solution of 17.5 g of trifluoroacetic anhydride in 50 mL of tetrahydrofuran during 60 min and the mixture was continued to stirring overnight. After the reaction mixture was heated to reflux for 15 min, it was poured carefully into 500 mL of water. After stirring for 30 min, crystals were precipitated and collected by filtration and dried in air to obtain 5.7 g of 2-phenyl-4-trichloromethyl-6-trifluoromethy-s-triazine as white crystals. Recrystallization from acetonitrile yields 4.8 g of pure crystals.

¹³C NMR (176 MHz, CDCl₃) δ 94.66 (CCl₃), 118.52 (CF₃, q, 277.2 Hz), 129.28 (Ar), 130.15 (Ar), 132.97 (Ar), 135.17 (Ar), 166.30 (q, 39.0 Hz, C—CF₃), 174.99, 175.18 (C—Ar, C—CCl₃).

### Synthesis of Triazine 2: 2-(p-Fluorophenyl)-4-trichloromethyl-6-trifluoromethy-s-triazine

Step a

To a solution of 10 g of p-fluorobenzamidine hydrochloride and 100 mL of Methanol, 10.3 g of 30% solution of NaOMe were added drop wise with stirring at zero degree. After addition, the mixture was stirred for 30 min, then 8.23 g of trichloroacetonitrile were added at zero degree during 30 min. After the addition, the cooling bath was removed and the reaction mixture was continued to stir overnight. 100 mL of ethyl acetate was added to the reaction mixture and the solid that precipitated in the flask was removed by filtration. The filtered solution was evaporated to yield N-(p-fluorobenzamidyl) trichloroacetamidine as oil. Yields 11.2 g.

Step b

10.0 g of N-(p-fluorobenzamidyl) trichloroacetamidine in 50 mL of tetrahydrofuran were added drop wise carefully to a cooled solution of 16.35 g of trifluoroacetic anhydride in 50 mL of tetrahydrofuran during 60 min and the mixture was continued to stirring overnight. After the reaction mixture was heated to reflux for 15 min, it was poured carefully into 500 mL of water. After stirring for 30 min, crystals were precipitated and collected by filtration. Recrystallization from methanol yielded 7.9 g of pure crystals of 2-(p-fluorophenyl)-4-trichloromethyl-6-trifluoromethy-s-triazine.

¹³C NMR (176 MHz, CDCl₃) δ 94.56 (CCl₃), 116.61 (Ar), 116.74 (Ar), 118.46 (CF₃, q, 277 Hz), 129.26 (Ar), 132.84 (Ar), 166.33 (q, 39.2 Hz, C—CF₃), 167.33 (d, 257.8 Hz, Ar—F), 173.88, 175.22 (C—Ar, C—CCl₃).

### Synthesis of Triazine 3: 2-(p-Methylphenyl)-4-trichloromethyl-6-trifluoromethy-s-triazine

Step a

To a solution of 10.0 g of p-methylbenzamidine hydrochloride and 100 mL of Methanol, 10.3 g of 30% solution of NaOMe were added drop wise with stirring at zero degree. After addition, the mixture was stirred for 30 min, then 8.46 g of trichloroacetonitrile were added at zero degree during 30 min. After the addition, the cooling bath was removed and the reaction mixture was continued to stir overnight. 100 mL of ethyl acetate was added to the reaction mixture and the solid that precipitates in the flask was removed by filtration. The filtered solution was evaporated to yield N-(p-methylbenzamidyl) trichloroacetamidine as oil. Yields 18.8 g.

Step b

10.0 g of N-(p-methylbenzamidyl) trichloroacetamidine in 50 mL of tetrahydrofuran were added drop wise carefully to a cooled solution of 16.59 g of trifluoroacetic anhydride in 50 mL of tetrahydrofuran during 60 min and the mixture was continued to stirring overnight. After the reaction mixture was heated to reflux for 15 min, it was poured carefully into 500 mL of water. After stirring for 30 min, the water solution was extracted with 300 mL of ethyl acetate. The organic layer was separated and evaporated to give oil. After column chromatography with silica gel (cyclohexane/ethyl acetate=16/5), 2-(p-methylphenyl)-4-trichloromethyl-6-trifluoromethy-s-triazine as pure solid. Yields 1.4 g.

¹³C NMR (176 MHz, CDCl₃) δ 22.00 (CH₃), 94.74 (CCl₃), 118.54 (CF₃, q, 278 Hz), 130.06 (Ar), 130.20 (Ar), 130.35 (Ar), 146.55 (CH₃—Ar), 166.15 (q, 38.7 Hz, C—CF₃), 174.87, 174.98 (C—Ar, C—CCl₃).

### Synthesis of Triazine 4: 2-(p-methoxylphenyl)-4-trichloromethyl-6-trifluoromethy-s-triazine

Step a

To a solution of 10.0 g of p-methoxybenzamidine hydrochloride and 100 mL of Methanol, 10.3 g of 30% solution of NaOMe were added dropwise with stirring at zero degree. After addition, the mixture was stirred for 30 min, then 7.73 g of trichloroacetonitrile were added at zero degree during 30 min. After the addition, the cooling bath was removed and the reaction mixture was continued to stir overnight. 100 mL of ethyl acetate was added to the reaction mixture and the solid that precipitates in the flask was removed by filtration. The filtered solution was evaporated to yield N-(p-methoxylbenzamidyl) trichloroacetamidine as oil. Yields 15.9 g.

Step b

10.0 g of N-(p-methoxylbenzamidyl) trichloroacetamidine in 50 mL, of tetrahydrofuran were added drop wise carefully to a cooled solution of 16.69 g of trifluoroacetic anhydride in 50 mL of tetrahydrofuran during 60 min and the mixture was continued to stirring overnight. After the reaction mixture was heated to reflux for 15 min, it was poured carefully into 500 mL of water. After stirring for 30 min, the water solution was extracted with 300 mL, of ethyl acetate. The organic layer was separated and evaporated to give oil. After column chromatography with silica gel (Cyclohexane/Ethyl acetate=16/5), 2-(p-methoxylphenyl)-4-trichloromethyl-6-trifluoromethy-s-triazine as pure solid. Yields 3.5 g.

¹³C NMR (176 MHz, CDCl₃) δ 55.70 (OCH₃), 114.70 (Ar), 118.58 (CF₃, q, 277 Hz), 125.49 (Ar), 132.52 (Ar), 165.52 (CH₃—Ar), 166.00 (q, 38.7 Hz, C—CF₃), 174.20, 174.77 (C—Ar, C—CCl₃).

Measurement of Photosensitivity:

### Example 1

Photosensitive formulation was prepared by mixing 2 mg of Safranine O, 20 mg of CGI909, 20 mg of triazine 1, 200 mg of DMSO, 2.0 g of SR 349 and stirring overnight to ensure complete mixing. The photosensitive formulation was coated with a thickness of 25 μm onto a polyethylene film and covered with a further polyethylene film protect against oxygen from air. Then the sample was measured by Real-Time FTIR and the result is shown in FIG. 3. The polymerization conversion after 40 sec irradiation was 53.2%.

### Example 2

Example 2 was performed using the same procedure as example 1 using triazine 2, instead of triazine 1. The polymerization conversion after 40 sec irradiation was 53.5%.

### Example 3

Example 3 was performed using the same procedure as example 1 using triazine 3, instead of triazine 1. The polymerization conversion after 40 sec irradiation was 53.5%.

### Comparative Example 1

Comparative example 1 was performed using the same procedure as example 1 using commercially available Triazine-A instead of Triazine 1. Conversion during irradiation was determined using Real-Time FTIR and results are shown in FIG. 3. The polymerization conversion after 40 sec irradiation was 49.8%.

TABLE 1
Result of conversion in photopolymerisation experiment

| | | Conversion after 40 s |
|---|---|---|
| Example | Triazine | [%] |
| 1 | 1 | 53.2 |
| 2 | 2 | 53.5 |
| 3 | 3 | 53.5 |
| Comparative Example 1 | Triazine-A | 49.8 |

The examples 1-3 show a higher conversion rate after 40 s than the comparative example 1 thus demonstrating the higher efficiency of the triazines 1-3 compared to triazine A. In a direct comparison, example 1 shows a higher conversion rate than the comparative example 1 during the whole recordings period (FIG. 3).

Preparation of Photopolymer Compounds:

### Preparation of Polyol 1

In a 1 L flask, 0.18 g of tin octoate, 374.8 g of ε-caprolactone and 374.8 g of a difunctional polytetrahydrofuran polyetherpolyol (equivalent weight 500 g/mol of OH) were initially charged and heated up to 120° C. and maintained at that temperature until the solids content (proportion of nonvolatile constituents) was 99.5% by weight or higher. This was followed by cooling to obtain the product as a waxy solid.

### Preparation of Acrylate 1: (phosphorus thioyltris(oxy-4,1-phenyleneiminocarbonyloxy-ethane-2,1-diyl) triacrylate)

In a 500 mL round-bottom flask, 0.1 g of 2,6-di-tert-butyl-4-methylphenol, 0.05 g of dibutyltin dilaurate (Desmorapid® Z, Bayer MaterialScience AG, Leverkusen, Germany) and also and 213.07 g of a 27% solution of tris(p-isocyanatophenyl) thiophosphate in ethyl acetate (Desmodur® RFE, product from Bayer MaterialScience AG, Leverkusen, Germany) were initially charged and heated to 60° C. Thereafter, 42.37 g of 2-hydroxyethyl acrylate were added dropwise and the mixture was further maintained at 60° C. until the isocyanate content had dropped below 0.1%. This was followed by cooling and complete removal of the ethyl acetate under reduced pressure to obtain the product as a partly crystalline solid.

### Preparation of Acrylate 2: 2-({[3-(methylsulphanyl)phenyl]carbamoyl}oxy)ethyl prop-2-enoate)

In a 100 mL round-bottom flask, 0.02 g of 2,6-di-tert-butyl-4-methylphenol, 0.01 g of Desmorapid® Z, 11.7 g of 3-(methylthio)phenyl isocyanate were initially charged and heated to 60° C. Thereafter, 8.2 g of 2-hydroxyethyl acrylate were added dropwise and the mixture was further maintained at 60° C. until the isocyanate content had dropped below 0.1%. This was followed by cooling to obtain the product as a pale yellow liquid.

### Preparation of Additive 1: (Bis(2,2,3,3,4,4,5,5,6,6,7,7-dodecafluoroheptyl) 2,2,4-trimethylhexane-1,6-diyl biscarbamate)

In a round-bottom flask, 0.02 g of Desmorapid Z and 3.6 g of 2,4,4-trimethylhexanes 1,6-diisocyanate were initially charged and heated to 70° C. This was followed by the dropwise addition of 11.39 g of 2,2,3,3,4,4,5,5,6,6,7,7-dodecafluoroheptan-1-ol and the mixture was further maintained at 70° C. until the isocyanate content had dropped below 0.1%. This was followed by cooling to obtain the product as a colorless oil.

Preparation of Holographic Media:

Example Medium 1 (M1)

3.38 g of polyol component 1 were mixed with 2.00 g of acrylate 1, 2.00 g of acrylate 2, 1.50 g of additive 1, 0.10 g of CGI 909 (product from BASF SE, Basle, Switzerland), 0.018 g of Dye 1, 0.09 g of example 1 and 0.35 g of ethyl acetate at 40° C. to obtain a clear solution. The solution was then cooled down to 30° C., 0.65 g of Desmodur® N3900 (commercial product from Bayer MaterialScience AG, Leverkusen, Germany, hexane diisocyanate-based polyisocyanate, portion on iminooxadiazinedione at least 30%, NCO content: 23.5%) was added before renewed mixing. Finally, 0.01 g of Fomrez UL 28 (urethanization catalyst, commercial product of Momentive Performance Chemicals, Wilton, Conn., USA) was added and again briefly mixed in. The mixed photopolymer formulation was applied on 36 μm thick polyethylene terephthalate film. The coated film was dried for 5.8 minutes at 80° C. and finally covered with a 40 μm polyethylene film. The achieved photopolymer layer thickness was around 14 μm.

Example medium 2 (M2) was prepared as described above but with 0.09 g of example 3 instead of example 1.

Reference medium (RM 1) was prepared as described above but with 0.09 g of triazine A instead of example 1. The media obtained as described were subsequently tested for their holographic properties in the manner described above using a measuring arrangement as FIG. 1. The following measurements were obtained for Δn at dose E [mJ/cm²]:

TABLE 2
Result of holographic experiment

| | | Laser used to | | | |
|---|---|---|---|---|---|
| | | record Hologram | | | Dose |
| Example | Triazine | (nm) | DE | Δn | (mJ/cm²) |
| M1 | 1 | 532 | 0.84 | 0.024 | 31.8 |
| M2 | 3 | 532 | 0.85 | 0.023 | 31.8 |
| RM 1 | Triazine-A | 532 | 0.84 | 0.022 | 31.8 |

The above experimental data shows that the inventive photopolymer is useful to write bright holograms.

1.-14. (canceled)

15. A triazine photoinitiator of the formula (I) 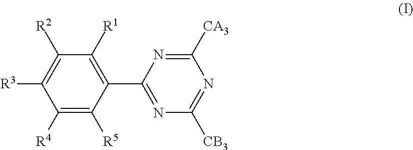
in which
A represents chlorine,
B represents fluorine,
R¹-R⁵ independently represent hydrogen, halogen alkyl, alkoxy, alkenyl, alkynyl, alkylthio, alkylseleno, nitro group with R¹ and R² and/or R² and R³ and/or R³ and R⁴ and/or R⁴ and R⁵ optionally form a 3 to 5 membered saturated or unsaturated ring which is optionally substituted with up to 2 hetero atoms and/or COOR⁶, COR⁷, CONHR⁸ radicals, whereby
R⁶, R⁷, R⁸ all independently from one another represent hydrogen, halogen and/or C1-C10-alkyl and/or C₁-C₁₀-alkoxy-substituted linear C₅-C₂₀-alkyl, in which up to 6 carbon atoms may be substituted with oxygen with the prerequisite that each two oxygen atoms are entangled by at least 2 carbon atoms and R⁶, R⁷, R⁸ start with at least 2 carbon atoms and the terminal group of the C₅-C₂₀-alkyl group is a methyl group.

16. Triazine photoinitiator according to claim 15, wherein R³ represents a hydrogen, methyl, halogene, methoxy, cyano, carboxylate, nitro or a methyl trihalogeno radical, while R¹, R², R⁴ and R⁵ independently represent hydrogen, halogen, alkyl, alkoxy, alkenyl, alkynyl, alkylthio, alkylseleno, nitro group with R¹ and R² and/or R² and R³ and/or R³ and R⁴ and/or R⁴ and R⁵ optionally form a 3 to 5 membered saturated or unsaturated ring which is optionally substituted with up to 2 hetero atoms and/or COOR⁶, COR⁷, CONHR⁸ radicals while R⁶, R⁷, R⁸ represent all independently from one another hydrogen, halogen and/or C₁-C₁₀-alkyl and/or C₁-C₁₀-alkoxy-substituted linear C₅-C₂₀-alkyl, in which up to 6 carbon atoms may be substituted with oxygen with the prerequisite that each two oxygen atoms are entangled by at least 2 carbon atoms and R⁶, R⁷, R⁸ start with at least 2 carbon atoms and the terminal group of the C₅-C₂₀-alkyl group is a methyl group.

17. Triazine photoinitiator according to claim 15, wherein R¹, R², R⁴ and R⁵ represent a hydrogen atom.

18. Triazine photoinitiator according to claim 15, wherein R¹, R², R⁴ and R⁵ represent a hydrogen atom and R³ represents a hydrogen atom, a methyl, fluorine or methoxy radical.

19. Process for the preparation of a triazine according to claim 15 comprising the steps of
a) Reacting the respective benzamidine hydrochloride of formula (II) with trihalogenoacetonitrile in the presence of a catalyst and 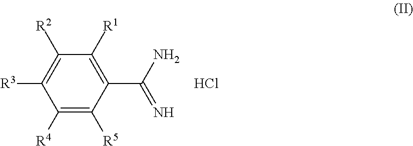
b) Reacting the resulting N-(benzamidyl) trihalogenoacetamidine with trihalogenoacetic anhydride,
wherein the radicals of R¹ to R⁵ are those as defined for formula (I) in claim 15, whereby the trihalogenoacetonitrile of a) carries three halogen atom different from the three halogen atoms of the trihalogenoacetic anhydride of b);
wherein the benzamidine hydrochloride is reacted with trichloroacetonitrile and the resulting N-(benzamidyl) trichloroacetamidine is reacted with trifluoroacetic anhydride.

20. (canceled)

21. Photopolymer composition comprising a photopolymerizable component and a photo initiator system, wherein the photo initiator system comprises a triazine according to claim 15.

22. Photopolymer composition according to claim 21, wherein it comprises 0.01 to 20 weight of the triazine.

23. Photopolymer composition according to claim 21, wherein the photo initiator system further comprises at least one co-initiator, selected from borate initiators, trichloromethyl initiators, aryloxide initiators, bisimidazole initiators, ferrocene initiators, aminoalkyl initiators, oxime initiator, thiol initiators, peroxide intiators.

24. Photopolymer composition according to claim 21, wherein it further comprises matrix polymers.

25. Photopolymer composition according to claim 24, wherein the matrix polymers are three dimensional cross-linked.

26. Holographic media wherein it comprises a photopolymer composition according to claim 21.

27. Hologram comprising a holographic medium according to claim 26.

28. (canceled)
